(19) United States
(12) Patent Application Publication
(10) Pub. No.: US 2024/0350016 A1
(43) Pub. Date: Oct. 24, 2024

(54) System and Method for Placement of a Central Catheter Tip

(71) Applicant: Bard Access Systems, Inc., Salt Lake City, UT (US)

(72) Inventor: Robin Scott Urry, Syracuse, UT (US)

(73) Assignee: Bard Access Systems, Inc., Salt Lake City, UT (US)

(21) Appl. No.: 18/136,063

(22) Filed: Apr. 18, 2023

(51) Int. Cl. A61B 5/00

(52) U.S. Cl. CPC A61B 5/0084; A61B 5/6852; A61B 2562/02

(57) ABSTRACT

A medical system includes a vascular device operatively coupled with a system module. The vascular device includes a pressure measurement capability as the distal end thereof. Logic of the system module acquires and pressure measurements during downstream advancement of the vascular device along a venous vasculature. The logic determines that the distal end of the vascular device is advanced to a defined location, such as the cavoatrial junction, for example, based on a minimum average pressure and/or maximum pressure variation. The vascular device may include a pressure sensor located at the distal end. The pressure sensor may include fiber optic Bragg grating of an optical fiber extending along the vascular device. The vascular device may be central catheter.

### BACKGROUND

The placement of the catheters such as central catheters commonly requires the catheter tip to be placed at a specific location within the patient vasculature, such as the lower one-third of the superior vena cava or the cavoatrial junction, for example. Misplacement of the catheter tip may result in risk to the patient or reduced effectiveness of the catheter procedure. In some instances of misplacement, replacement of the catheter may be required resulting in further risk to the patient and increased medical expense.

Disclosed herein are systems, devices, and methods that enable the placement of a catheter tip at a defined location within the patient vasculature.

### SUMMARY

Disclosed herein is a medical system that, according to some embodiments, includes an elongate medical device configured for advancement along a vasculature of a patient, where the elongate medical device includes a pressure sensor coupled therewith, and where the pressure sensor is configured to determine a pressure adjacent a distal end of the elongate medical device. The system further includes a system module that includes a console coupled with the pressure sensor. The console includes a processor and a memory having logic stored thereon that, when executed by the processor, performs operations of the system. The operations include (i) receiving a plurality of pressure measurements from the pressure sensor during advancement of the elongate medical device along the vasculature, (ii) determining a trend of a pressure parameter of the plurality of pressure measurements with respect to positioning of the distal end within the vasculature, and (iii) determining a position of the distal end within the vasculature based on the trend of the pressure parameter.

In some embodiments, the pressure sensor is located adjacent the distal end of the elongate medical device. In some embodiments, the elongate medical device includes an optical fiber extending therealong, and the pressure sensor includes a fiber optic Bragg grating.

In some embodiments, the elongate medical device is a central catheter, and in some embodiments, the vasculature includes a vein. In such embodiments, advancement includes advancement toward a heart of the patient, and in some embodiments, the position of the distal end within the vasculature includes a cavoatrial junction.

In some embodiments, the pressure parameter includes an average pressure of a subset of the plurality of pressure measurements, and the trend includes a decrease in the average pressure as the elongate medical device is advanced. In such embodiments, determining a position of the distal end within the vasculature based on the trend includes determining a minimum of the average pressure in accordance with the trend.

In some embodiments, the pressure parameter includes a pressure variation of a subset of the plurality of pressure measurements, and the trend includes an increase in the pressure variation as the elongate medical device is advanced. In such embodiments, determining a position of the distal end within the vasculature based on the trend includes determining a maximum of the pressure variation in accordance with the trend.

In some embodiments, the pressure parameter includes (i) an average pressure of a subset of the plurality of pressure measurements, where the trend includes a decrease in the average pressure; and (ii) a pressure variation of the subset of the plurality of pressure measurements, where the trend further includes an increase in the pressure variation. Further in such embodiments, determining the position of the distal end within the vasculature based on the trend includes determining a minimum of the average pressure in combination with determining a maximum of the pressure variation.

Also disclosed herein is a method of placing a catheter within a vasculature that, according to some embodiments, includes (i) obtaining a plurality of pressure measurements adjacent a distal end of the catheter during advancement toward a defined final position of the distal end, (ii) determining a trend of a pressure parameter of the plurality of pressure measurements with respect to positioning of the distal end within the vasculature during advancement, and (iii) determining that the distal end is located at the defined final position based on the trend.

In some embodiments of the method, a pressure sensor coupled with the catheter is configured to obtain the plurality of pressure measurements.

In some embodiments of the method, the catheter is a central catheter and the defined final position is a cavoatrial junction.

In some embodiments of the method, the pressure parameter includes an average pressure of a subset of the plurality of pressure measurements, and the trend includes a decrease in the average pressure. In such embodiments of the method, determining that the distal end is located at the defined final position based on the trend includes determining a minimum of the average pressure in accordance with the trend.

In some embodiments of the method, the pressure parameter includes a pressure variation of a subset of the plurality of pressure measurements, and the trend includes an increase in the pressure variation. In such embodiments of the method, determining that the distal end is located at the defined final position based on the trend includes determining a maximum of the pressure variation in accordance with the trend.

In some embodiments of the method, the pressure parameter includes an average pressure of a subset of the plurality of pressure measurements, and the trend includes a decrease in the average pressure. The pressure parameter further includes a pressure variation of the subset of the plurality of pressure measurements, and the trend further includes an increase in the pressure variation. In such embodiments of the method, determining that the distal end is located at the defined final position based on the trend includes determining a minimum of the average pressure in combination with determining a maximum of the pressure variation.

In some embodiments of the method, the pressure sensor is located adjacent the distal end of the catheter. Further in some embodiments of the method, the catheter includes an optical fiber extending therealong, and the pressure sensor includes a fiber optic Bragg grating.

These and other features of the concepts provided herein will become more apparent to those of skill in the art in view of the accompanying drawings and following description, which describe particular embodiments of such concepts in greater detail.

### DRAWINGS

FIG. 1 illustrates a medical system including an elongate medical device in use with a patient, in accordance with some embodiments.

FIG. 2 is a representative pressure chart illustrating parameters of a venous pressure along a venous vasculature, in accordance with some embodiments.

FIGS. 3A-3D illustrate different embodiments of the elongate medical device of FIG. 1, in accordance with some embodiments.

FIG. 4 illustrates a block diagram of a method of placing a catheter, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The phrases “connected to,” “coupled with,” and “in communication with” refer to any form of interaction between two or more entities, including but not limited to mechanical, electrical, magnetic, electromagnetic, fluid, and thermal interaction. Two components may be coupled with each other even though they are not in direct contact with each other. For example, two components may be coupled with each other through an intermediate component.

The terms “proximal” and “distal” refer to opposite ends of a medical device, including the devices disclosed herein. As used herein, the proximal portion of a medical device is the portion nearest a practitioner during use, while the distal portion is the portion at the opposite end. For example, the distal end of medical device is defined as the end closest to the patient or furthest inserted into the patient during utilization of the medical device. The proximal end is the end opposite the distal end.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

Unless defined otherwise, all technical and scientific terms used herein have the same meaning as commonly understood by those of ordinary skill in the art. References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

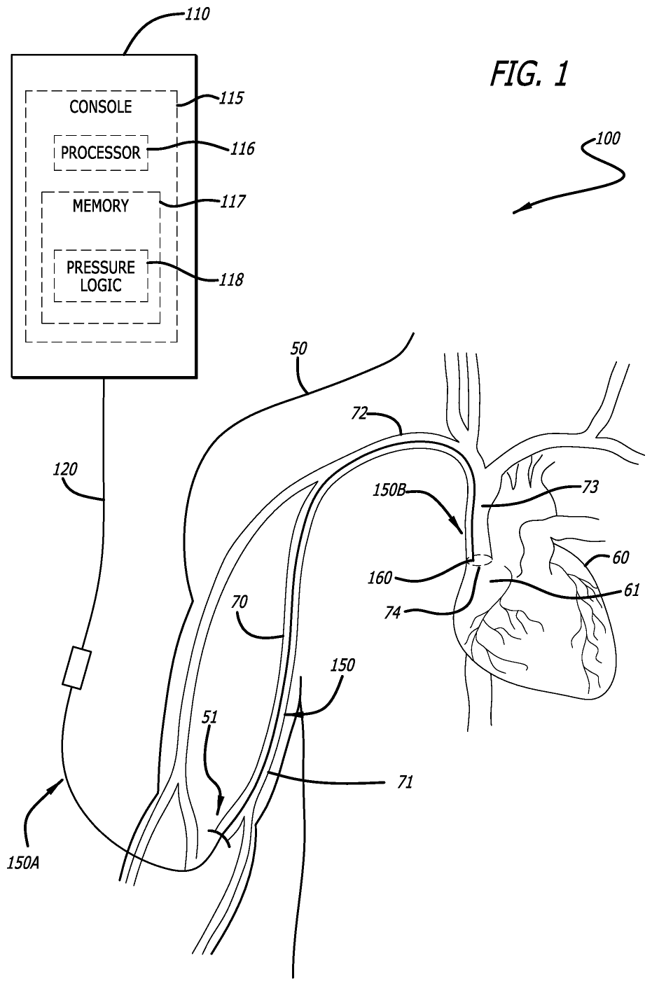

FIG. 1 illustrates medical system (system) 100 in use with a patient 50, in accordance with some embodiments. The system 100 is generally configured to enable a clinician to place a distal end of an elongated medical device (e.g., a central catheter) at a defined location within a vasculature of the patient 50. The system 100 generally includes the elongate medical device (device) 150 operatively coupled with a system module 110 having a console 115. A connecting member 120 operatively couples the device 150 with the system module 110.

FIG. 1 further illustrates various exemplary elements of the patient 50. A heart 60 of the patient 50 includes a right atrium 61. A venous vasculature 70 includes a brachial vein 71, a subclavian vein 72 and a superior vena cava 73. The superior vena cava 73 couples with the right atrium 61 at the cavoatrial junction 74.

The device 150 is configured for insertion within the venous vasculature 70 and may include any suitable medical device, such as a catheter, a stylet or guidewire, for example. In the illustrated embodiment, the device 150 is inserted into the brachial vein 71 at the insertion site 51. The device 150 extends along the brachial vein 71, the subclavian vein 72, and the superior vena cava 73. A distal end 150B of the device 150 is located at the cavoatrial junction 74. During advancement of the device 150, the distal end 150B is displaced distally (i.e. toward the heart 60) along the brachial vein, along the subclavian vein 72, and along the superior vena cava 73 to the cavoatrial junction 74.

The device 150 includes a pressure sensor 160 configured to measure a venous pressure adjacent the distal end 150B so that the system module 110 can monitor the venous pressure adjacent the distal end 150B. In some embodiments, the pressure sensor 160 be located at the distal end 150B. As such, during advancement, the system module 110 can monitor the venous pressure along the brachial vein 71, along the subclavian vein 72, along the superior vena cava 73, and at the cavoatrial junction 74.

The console 115 includes a processor 116 and a memory 117 (e.g., a non-transitory computer-readable medium) having pressure logic 118 stored thereon. The pressure logic 118, when executed by the processor 116, performs operations of the system 100 as further described below.

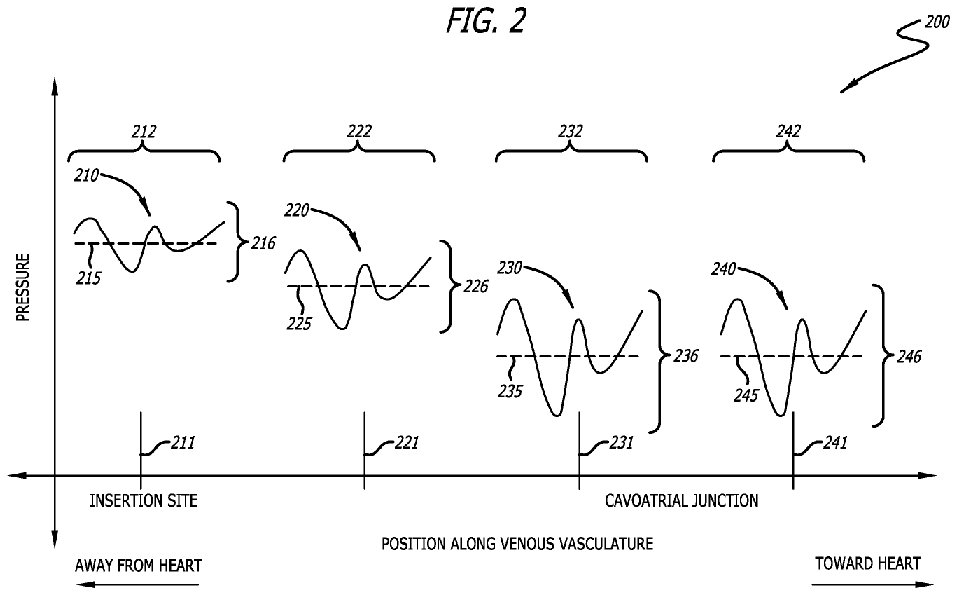

FIG. 2 illustrates a representative pressure chart 200 showing pressure on the Y-axis versus position along a venous vasculature on the X-axis, where the pressure may be measured by the pressure sensor 160, according to some embodiments. The pressure chart 200 illustrates venous pressure waves at different positions along the venous vasculature.

By way of one exemplary implementation, a first pressure wave 210 in accordance with a first position 211 (e.g., adjacent the insertion site) includes a first pressure measurement subset 212 (i.e., a number of pressure measurements obtained by the pressure sensor 160). The first pressure measurement subset 212 defines a first average pressure 215 and a first pressure variation 216 (e.g., standard deviation of the first pressure measurement subset 212). A second pressure wave 220 in accordance with a second position 221 located downstream of the first position 211 includes a second pressure measurement subset 222. The second pressure measurement subset 222 defines a second average pressure 225 and a second pressure variation 226. A third pressure wave 230 in accordance with a third position 231 located downstream of the second position 221 and at the entry of the cavoatrial junction 74 includes a third pressure measurement subset 232. The third pressure measurement subset 232 defines a third average pressure 235 and a third pressure variation 236. A fourth pressure wave 240 in accordance with a third position 241 located at the cavoatrial junction 74 includes a fourth pressure measurement subset 242. The fourth pressure measurement subset 242 defines a fourth average pressure 245 and a fourth pressure variation 246.

Consistent with fluid dynamic principles, a static pressure of a fluid within a flow conduit decreases along the direction of fluid flow. In other words, an upstream pressure is generally greater than a downstream pressure along a horizontal flow path. As applied to a venous vasculature, an average venous pressure along a vein (or venous vasculature) decreases along the vein in the direction of the heart. As applied to the exemplary implementation set forth above, the first average pressure 215 is greater than the second average pressure 225, and the second average pressure 225 is greater than the third average pressure 235.

Consistent with further fluid dynamic principles, a rate of decease in static pressure along the flow conduit is related to a velocity of the fluid flow, where a higher flow velocity is consistent with a higher rate of decreasing static pressure. In many fluid flow applications, a larger flow area (e.g., diameter of a flow conduit) is associated with a slower velocity for a given volumetric flow rate. As such, a rate of decrease in static pressure along a flow path having a larger flow area may be less than a rate of decrease in the static pressure along a flow path having a smaller flow area. As applied to the exemplary implementation set forth above, the flow area of the cavoatrial junction (i.e., the flow areas of the third position 231 and the fourth position 241) is large in relation to the flow areas of the first position 211and the second position 221. As such, a difference between the third average pressure 235 and the fourth average pressure 245 may be small, i.e., the third average pressure 235 and the fourth average pressure 245 may be substantially equal.

During advancement of the device 150 along the venous vasculature 70 toward the cavoatrial junction 74, the pressure logic 118 may perform operations related to the pressure within the venous vasculature 70. According to an exemplary instance of use, upon insertion of the device 150 through the insertion site 51 and into the venous vasculature 70, such that the distal end 150B is disposed at the first location 211, for example, the pressure logic 118 may receive the first pressure measurement subset 212 and calculate the first average pressure 215. Upon further insertion of the device 150, such that the distal end 150B is disposed at the second location 221, the pressure logic 118 may receive the second pressure measurement subset 222 and calculate the second average pressure 225. The pressure logic 118 may compare the second average pressure 225 with the first average pressure 215, and if the second average pressure 225 is less than the first average pressure 215 (indicating a decreasing trend in average pressure during advancement), the pressure logic 118 may determine that the device 150 is not sufficiently advanced to locate distal end 150B at the cavoatrial junction 74.

In further accordance with the exemplary instance of use set forth above, upon further insertion of the device 150, such that the distal end 150B is disposed at the third location 231, the pressure logic 118 may receive the third pressure measurement subset 232 and calculate the third average pressure 235. The pressure logic 118 may compare the third average pressure 235 with the second average pressure 225, and if the third average pressure 235 is less than the second average pressure 225 (indicating a continued decreasing trend in average pressure during advancement between the second position 221 and the third position 231), the pressure logic 118 may determine the device 150 is still not sufficiently advanced to the locate distal end 150B at the cavoatrial junction 74.

In further accordance with the exemplary instance of use set forth above, upon further insertion of the device 150, such that the distal end 150B is disposed at the fourth location 241, the pressure logic 118 may receive the fourth pressure measurement subset 242 and calculate the fourth average pressure 245. The pressure logic 118 may compare the fourth average pressure 245 with the third average pressure 235, and if the fourth average pressure 245 is substantially equal to the third average pressure 235 (indicating a non-decreasing or a flat trend in the average pressure during advancement between the third position 231 and the fourth position 241), the pressure logic 118 may determine that the device 150 is sufficiently advanced to the locate distal end 150B at the cavoatrial junction 74. As the third average pressure 235 and the fourth average pressure 245 are substantially equal, the pressure logic 118 may determine the third and fourth average pressures 235, 245 define a minimum venous pressure along the venous vasculature 70.

The heart 60 generates pressure pulses within the venous vasculature 70, and the magnitude of the pressure pulses (i.e., a pressure variation) changes along the venous vasculature 70. More specifically, the magnitude of the pressure pulses is maximum at the heart 60 and decreases with distance away from the heart 60 as illustrated in FIG. 2. During advancement of the device 150 along the venous vasculature 70 toward the cavoatrial junction 74, the pressure logic 118 may perform operations related to the pressure variation within the venous vasculature 70. According to an exemplary instance of use, upon insertion of the device 150 through the insertion site 51 and into the venous vasculature 70, such that the distal end 150B is disposed at the first location 211, the pressure logic 118 may receive the first pressure measurement subset 212 and calculate the first pressure variation 216 (e.g., standard deviation). Upon further insertion of the device 150, such that the distal end 150B is disposed at the second location 221, the pressure logic 118 may receive the second pressure measurement subset 222 and calculate the second pressure variation 226. The pressure logic 118 may compare the second pressure variation 226 with the first pressure variation 225, and if the second pressure variation 226 is greater than the first pressure variation 216 (indicating an increasing trend in pressure variation during advancement), the pressure logic 118 may determine that the device 150 is not sufficiently advanced to the locate distal end 150B at the cavoatrial junction 74.

In further accordance with the exemplary instance of use set forth above, upon further insertion of the device 150, such that the distal end 150B is disposed at the third location 231, for example, the pressure logic 118 may receive the third pressure measurement subset 232 and calculate the third pressure variation 236. The pressure logic 118 may compare the third pressure variation 236 with the second pressure variation 226, and if the third pressure variation 236 is greater than the second pressure variation 226 (indicating a continued increasing trend in pressure variation during advancement between the second position 221 and the third position 231), the pressure logic 118 may determine the device 150 is still not sufficiently advanced to the locate distal end 150B at the cavoatrial junction 74.

In further accordance with the exemplary instance of use set forth above, upon further insertion of the device 150, such that the distal end 150B is disposed at the fourth location 241, for example, the pressure logic 118 may receive the fourth pressure measurement subset 242 and calculate the fourth pressure variation 246. The pressure logic 118 may compare the fourth pressure variation 246 with the third pressure variation 236, and if the fourth pressure variation 246 is substantially equal to the third pressure variation 236 (indicating a none-increasing or flat trend in pressure variation during advancement between the third position 231 and the fourth position 241), the pressure logic 118 may determine that the device 150 is sufficiently advanced to the locate distal end 150B at the cavoatrial junction 74. As the third pressure variation 235 and the fourth pressure variation 245 are substantially equal, the pressure logic 118 may determine the third and fourth pressure variations 235, 245 define a maximum pressure variation of the venous pressure along the venous vasculature 70.

In some embodiments, the pressure logic 118 may combine the decreasing trend of the average pressure with the increasing trend of the pressure variation to determine the device 150 is sufficiently advanced to the locate distal end 150B at the cavoatrial junction 74. More specifically, the pressure logic 118 may, during advancement of the device 150, determine when the average pressure is at the minimum and determine when the pressure variation is at the maximum. The pressure logic 118 may then determine the device 150 is sufficiently advanced to the locate distal end 150B at the cavoatrial junction 74 when the average pressure is at the minimum and the pressure variation is at the maximum.

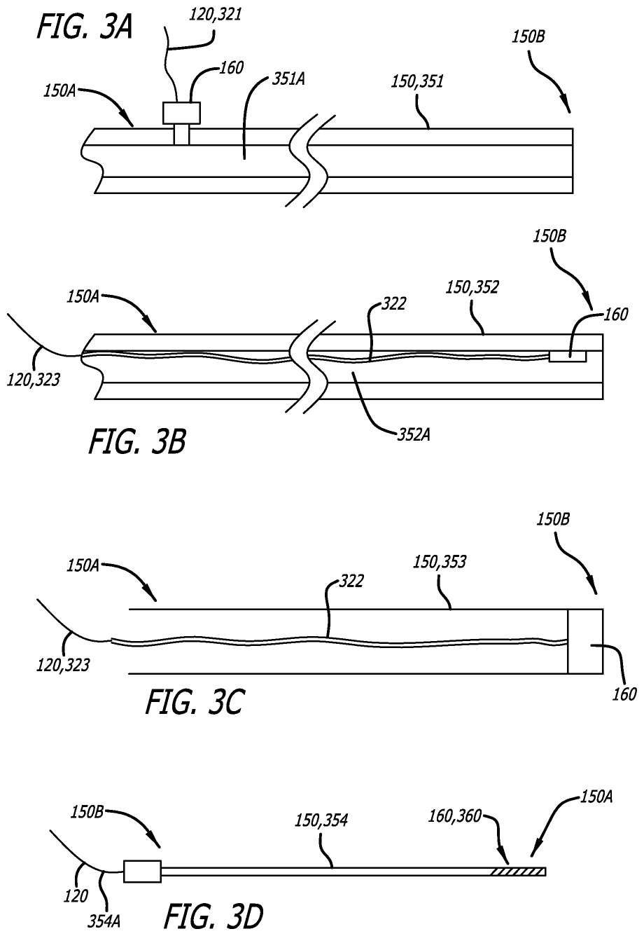

FIGS. 3A-3D illustrate different embodiments of the device 150, in accordance with some embodiments. FIG. 3A is illustrates a first embodiment of the device 150 that includes a catheter 351 defining a lumen 351A extending between the proximal end 150A and the distal end 150B. The pressure sensor 160 is coupled with the catheter 351 such that the pressure sensor 160 can measure a pressure within the lumen 351A. The pressure sensor 160 is located adjacent the proximal end 150A so that the pressure sensor 160 is disposed outside the patient 50 during use. The connecting member 120 includes a number of electrical leads 321 configured to electrically coupled the pressure sensor 160 with the console 115. During use, the lumen 351A defines a pressure connection between the pressure sensor 160 and the distal end 150B.

FIG. 3B is illustrates a second embodiment of the device 150 that includes a catheter 352 defining a lumen 352A extending between the proximal end 150A and the distal end 150B. The pressure sensor 160 is coupled with the catheter 351 at the distal end 150B such that the pressure sensor 160 can measure a pressure adjacent the distal end 150B. In various embodiments, the pressure sensor 160 may be located within the lumen 352A, embedded within a wall of the catheter 352, or attached to external surface of the catheter 352. Electrical leads 322 are coupled with the pressure sensor 160 and extend along the catheter 352. The connecting member 120 includes a number of electrical leads 323 configured to electrically couple the electrical leads 322 with the console 115.

FIG. 3C is illustrates a third embodiment of the device 150 that includes a stylet (or guidewire) 353 extending between the proximal end 150A and the distal end 150B. The pressure sensor 160 is coupled with the stylet 353 at the distal end 150B such that the pressure sensor 160 can measure a pressure adjacent the distal end 150B. In various embodiments, the pressure sensor 160 may be embedded within the cross section of the stylet 353 or attached to external surface of the stylet 353. Electrical leads 322 are coupled with the pressure sensor 160 and extend along the stylet 353. The connecting member 120 includes a number of electrical leads 323 configured to electrically couple the electrical leads 322 with the console 115.

FIG. 3D is illustrates a fourth embodiment of the device 150 that includes an optical fiber 354 extending between the proximal end 150A and the distal end 150B. The pressure sensor 160 includes a fiber optic Bragg grating 360 located at the distal end 150B, where the fiber optic Bragg grating 360 is configured define reflected optical signals based on a pressure adjacent the distal end 150B. In such embodiments, the system 100 includes a light source and an optical receiver optically coupled with the optical fiber 354. The connecting member 120 may include a connecting optical fiber 354A configured to couple the optical fiber 354 to the console 115.

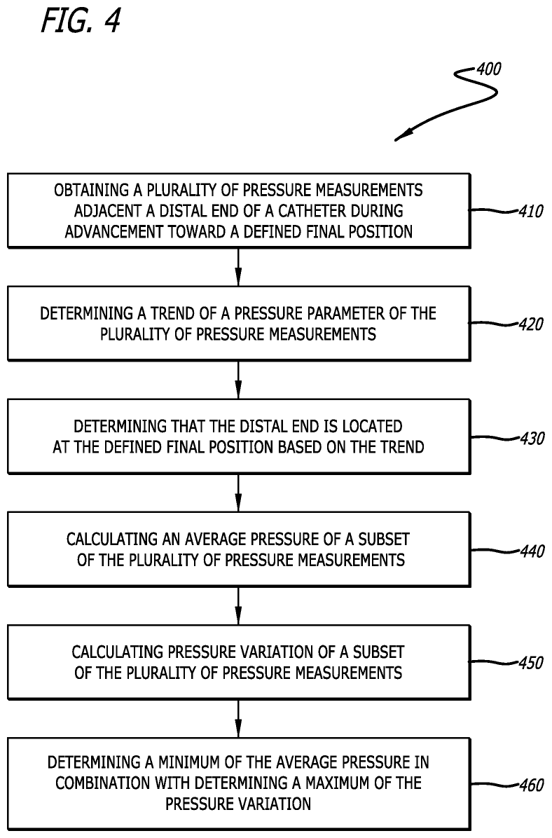

FIG. 4 illustrates a block diagram of a method of placing a catheter within a patient, in accordance with some embodiments. The method 400 may include all or any subset of the following actions, steps, or processes. The method 400 may include obtaining a plurality of pressure measurements adjacent a distal end of a catheter during advancement toward a defined final position of the distal end (block 410). The method 400 may further include determining a trend of a pressure parameter of the plurality of pressure measurements (block 420) with respect to positioning of the distal end within the vasculature during advancement. The method 400 may further include determining that the distal end is located at the defined final position based on the trend (block 430). In some embodiments of the method 400, a pressure sensor coupled with the catheter is configured to obtain the plurality of pressure measurements. In some embodiments of the method 400, the catheter is a central catheter and the defined final position is a cavoatrial junction

In some embodiments of the method 400, the pressure parameter includes an average pressure of a subset of the plurality of pressure measurements, and the trend includes a decrease in the average pressure. As such, the method 400 may including calculating an average pressure of a subset of the plurality of pressure measurements (block 440). In such embodiments of the method 400, determining that the distal end is located at the defined final position based on the trend includes determining a minimum of the average pressure in accordance with the trend.

In some embodiments of the method 400, the pressure parameter includes a pressure variation of a subset of the plurality of pressure measurements, and the trend includes an increase in the pressure variation. As such, the method 400 may including calculating a pressure variation of a subset of the plurality of pressure measurements (block 450). In such embodiments of the method 400, determining that the distal end is located at the defined final position based on the trend includes determining a maximum of the pressure variation in accordance with the trend.

In some embodiments of the method 400, the pressure parameter includes the average pressure and the pressure variation in combination. In such embodiments, the method 400 may further include determining a minimum of the average pressure in combination with determining a maximum of the pressure variation (block 460) to determining that the distal end is located at the defined final position based on the trend includes determining a minimum of the average pressure in combination with determining a maximum of the pressure variation.

In some embodiments of the method 400, the pressure sensor is located adjacent the distal end of the catheter. Further in some embodiments of the method 400, the catheter includes an optical fiber extending therealong, and the pressure sensor includes a fiber optic Bragg grating.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical system, comprising:
an elongate medical device configured for advancement along a vasculature of a patient, the elongate medical device comprising a pressure sensor coupled with the elongate medical device, wherein the pressure sensor is configured to determine a pressure adjacent a distal end of the elongate medical device; and
a system module including a console coupled with the pressure sensor, the console including a processor and a memory having logic stored thereon that, when executed by the processor, performs operations that include:
receiving a plurality of pressure measurements from the pressure sensor during advancement of the elongate medical device along the vasculature;
determining a trend of a pressure parameter of the plurality of pressure measurements with respect to positioning of the distal end within the vasculature during advancement; and
determining a position of the distal end within the vasculature based on the trend of the pressure parameter.

2. The system according to claim 1, wherein the pressure sensor is located adjacent the distal end of the elongate medical device.

3. The system according to claim 2, wherein:
the elongate medical device includes an optical fiber extending therealong, and
the pressure sensor includes a fiber optic Bragg grating.

4. The system according to claim 1, wherein the elongate medical device is a central catheter.

5. The system according to claim 1, wherein the vasculature includes a vein.

6. The system according to claim 1, wherein advancement includes advancement toward a heart of the patient.

7. The system according to claim 1, wherein the position of the distal end within the vasculature includes a cavoatrial junction.

8. The system according to claim 1, wherein:
the pressure parameter includes an average pressure of a subset of the plurality of pressure measurements, and
the trend includes a decrease in the average pressure.

9. The system according to claim 8, wherein determining a position of the distal end within the vasculature based on the trend includes determining a minimum of the average pressure in accordance with the trend.

10. The system according to claim 1, wherein:
the pressure parameter includes a pressure variation of a subset of the plurality of pressure measurements, and
the trend includes an increase in the pressure variation.

11. The system according to claim 10, wherein determining a position of the distal end within the vasculature based on the trend includes determining a maximum of the pressure variation in accordance with the trend.

12. The system according to claim 1, wherein:
the pressure parameter includes an average pressure of a subset of the plurality of pressure measurements, the trend including a decrease in the average pressure;
the pressure parameter further includes a pressure variation of the subset of the plurality of pressure measurements, the trend further including an increase in the pressure variation; and
determining the position of the distal end within the vasculature based on the trend includes determining a minimum of the average pressure in combination with determining a maximum of the pressure variation.

13. A method of placing a catheter within a vasculature, comprising:
obtaining a plurality of pressure measurements adjacent a distal end of the catheter during advancement toward a defined final position of the distal end;
determining a trend of a pressure parameter of the plurality of pressure measurements with respect to positioning of the distal end within the vasculature during advancement; and
determining that the distal end is located at the defined final position based on the trend

14. The method according to claim 13, wherein a pressure sensor coupled with the catheter is configured to obtain the plurality of pressure measurements.

15. The method according to claim 13, wherein the catheter is a central catheter and the defined final position is a cavoatrial junction

16. The method according to claim 13, wherein:
the pressure parameter includes an average pressure of a subset of the plurality of pressure measurements,
the trend includes a decrease in the average pressure, and
determining that the distal end is located at the defined final position based on the trend includes determining a minimum of the average pressure in accordance with the trend.

17. The method according to claim 13, wherein:
the pressure parameter includes a pressure variation of a subset of the plurality of pressure measurements,
the trend includes an increase in the pressure variation, and
determining that the distal end is located at the defined final position based on the trend includes determining a maximum of the pressure variation in accordance with the trend.

18. The method according to claim 13, wherein:
the pressure parameter includes an average pressure of a subset of the plurality of pressure measurements, the trend including a decrease in the average pressure;
the pressure parameter further includes a pressure variation of the subset of the plurality of pressure measurements, the trend further including an increase in the pressure variation; and
determining that the distal end is located at the defined final position based on the trend includes determining a minimum of the average pressure in combination with determining a maximum of the pressure variation.

19. The method according to claim 14, wherein the pressure sensor is located adjacent the distal end of the catheter.

20. The method according to claim 19, wherein:
the catheter includes an optical fiber extending therealong, and
the pressure sensor includes a fiber optic Bragg grating.
